(19) United States
(12) Patent Application Publication
(10) Pub. No.: US 2020/0337676 A1
(43) Pub. Date: Oct. 29, 2020

(54) ULTRASONIC APPARATUS, AND METHOD FOR CONTROLLING THE SAME

(71) Applicant: General Electric Company, Schenectady, NY (US)

(72) Inventors: Naohisa KAMIYAMA, Hino, Tokyo (JP); Takuma OGURI, Hino, Tokyo (JP)

(21) Appl. No.: 16/852,813

(22) Filed: Apr. 20, 2020

(30) Foreign Application Priority Data
Apr. 26, 2019 (JP) 2019-085740

(51) Int. Cl. A61B 8/00; A61B 8/08

(52) U.S. Cl. CPC A61B 8/4444; A61B 8/5207

(57) ABSTRACT

A processor in an ultrasonic diagnostic apparatus controls an ultrasonic probe 2 to transmit a first ultrasonic beam to a first region A1, transmit a second ultrasonic beam to a second region A2, and transmit a third ultrasonic beam to a third region A3, wherein the first and third ultrasonic beams are focused ultrasonic beams having focus points F, and the second ultrasonic beam is an ultrasonic beam formed by a plane wave. The processor produces an ultrasonic image comprised of a first ultrasonic image for the first region A1, a second ultrasonic image for the second region A2, and a third ultrasonic image for the third region A3 based on first, second, and third echo signals obtained from said first, second, and third regions A1, A2, A3.

### CROSS-REFERENCE TO RELATED APPLICATIONS

This specification is based upon and claims the benefit of priority from Japanese patent application number JP 2019-085740 filed on Apr. 26, 2019, the entire contents of which are incorporated herein by reference.

### FIELD OF THE INVENTION

This disclosure relates to an ultrasonic apparatus for acquiring an ultrasonic image of a subject to be examined, and a method for controlling the same.

### BACKGROUND

Ultrasonic apparatuses transmit ultrasound from an ultrasonic probe to a subject to be examined, and produce an ultrasonic image from echo signals of the ultrasound. The ultrasonic probe has vibrating elements comprised of a plurality of channels, and transmission delays are applied thereto to set a focus at a specific depth, from which a focused ultrasonic beam is formed. On receipt, receive signals in a plurality of scan lines are acquired at one transmission by changing delays on received RF signals in several different manners. Even though the receive signals can be acquired outside of the region of a transmit sound field, such receive signals are unsatisfactory. This means the number of receive scan lines to be formed is thus limited by the profile of the transmit sound field.

Then, a technique called RTF (Retrospective Transmit Focus) method is used for forming more receive scan lines per transmission. This method transmits plane-wave ultrasound having a transmit focus set at infinity. The plane-wave ultrasound has a wider and more uniform transmit sound field than that of a focused ultrasonic beam. This enables forming more receive scan lines per transmission to significantly improve the frame rate.

The wide and uniform transmit sound field, however, has dispersed energy of transmitted ultrasound as compared with the focused transmit sound field; this lowers the S/N ratio for received signals. Thus, in the RTF method, a plurality of number of ultrasound transmissions are performed in producing an ultrasonic image in one frame, causing respective transmit sound fields formed by the plurality of number of ultrasound transmissions to overlap one another for one receive acoustic line. This allows to obtain a plurality of echo signals in one receive acoustic line and then the plurality of echo signals are superimposed (compounded) to thereby improve the S/N ratio. The plurality of number of ultrasound transmissions including one receive acoustic line causes low frame rate as compared with one ultrasound transmission for one receive acoustic line. On the other hand, the plane-wave ultrasound, which is one of the characteristics in the RTF method as described above, can improve frame rate by an increased beam width as compared with the case in which ultrasound having a focus is transmitted.

In the aforementioned RTF method, the number of transmissions is smaller for receive acoustic lines in an end portion of an image production region, resulting in a reduced number of compoundings of echo signals, and in turn, resulting in a reduced number of elements used in the transmission/reception. Accordingly, it is difficult to secure a sufficient S/N ratio in the end portion of the image production region, and thus, image quality may be disadvantageously lowered as compared with conventional methods based on a focused sound field.

### BRIEF SUMMARY OF THE INVENTION

An ultrasonic apparatus according to one aspect includes: an ultrasonic probe for transmitting ultrasonic beams to an image production region in a subject to be examined and acquiring echo signals, wherein said image production region is comprised of a first region, a second region, and a third region, said second region lying between said first region and said third region; and a processor for controlling transmission of said ultrasonic beams by said ultrasonic probe, and production of an ultrasonic image for said image production region based on said echo signals, wherein said processor controls said ultrasonic probe to transmit a first ultrasonic beam to said first region, transmit a second ultrasonic beam to said second region, and transmit a third ultrasonic beam to said third region, said first and third ultrasonic beams being focused ultrasonic beams, and said second ultrasonic beam being a plane wave, and produces said ultrasonic image comprised of a first ultrasonic image for said first region, a second ultrasonic image for said second region, and a third ultrasonic image for said third region based on first, second, and third echo signals obtained from said first, second, and third regions by transmissions of said first, second, and third ultrasonic beams.

According to the one aspect above, since first and third ultrasonic beams, which are focused ultrasound, are transmitted for first and third regions lying on both end sides of an image production region, deterioration in the S/N ratio encountered in the conventional RTF method can be improved. In addition, since a second ultrasonic beam formed by a plane wave is transmitted in a second region between the first and third regions, the frame rate can be improved as compared with the case in which focused ultrasonic beams are transmitted in the entire image production region.

### BRIEF DESCRIPTION OF THE DRAWINGS

FIG. 1 is a block diagram showing a configuration of an ultrasonic diagnostic apparatus in accordance with an embodiment.

FIG. 2 is a diagram showing an image production region.

FIG. 3 is a diagram showing first ultrasonic beams.

FIG. 4 is a diagram explaining the positions of focus points in first and third ultrasonic beams.

FIG. 5 is a diagram showing a plurality of second ultrasonic beams.

FIG. 6 is a flow chart of transmission of ultrasonic beams and production of an ultrasonic image.

FIG. 7 is a diagram explaining creation of raw data in receive acoustic lines in a second region.

FIG. 8 is a diagram showing a receive acoustic line, ultrasonic beams that are plane waves for acquiring raw data in the receive acoustic line, and widths of their transmit sound fields.

FIG. 9 is a diagram showing a receive acoustic line at a position different from that of the receive acoustic line in FIG. 8, ultrasonic beams that are plane waves for acquiring raw data in the receive acoustic line, and widths of their transmit sound fields.

FIG. 10 is a diagram showing first ultrasonic beams BM1 of which the positions of focus points are identical in an acoustic-line direction.

FIG. 11 is a diagram showing a plurality of focus points set in one transmit acoustic line.

### DETAILED DESCRIPTION OF THE INVENTION

Now an embodiment of the present invention will be described referring to the drawings. In the following embodiment, an ultrasonic diagnostic apparatus for displaying an ultrasonic image of a subject to be examined for the purpose of diagnosis, etc. will be addressed as an example of the ultrasonic apparatus in accordance with the present invention.

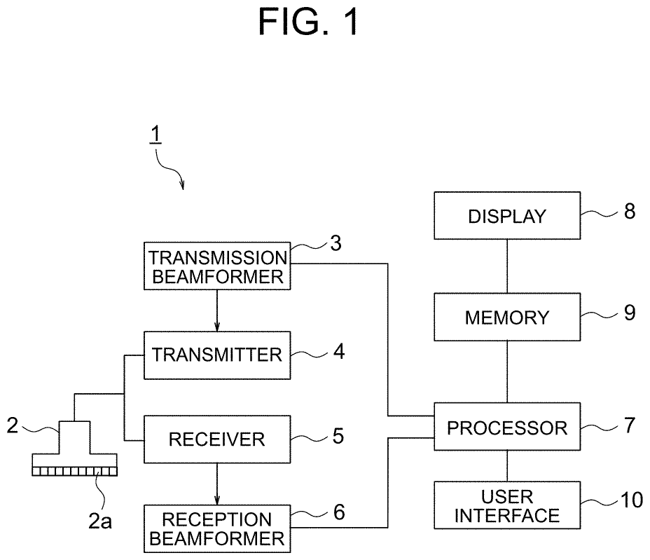

An ultrasonic diagnostic apparatus 1 shown in FIG. 1 includes a transmit beamformer 3 and a transmitter 4 that drive a plurality of vibrating elements 2a arranged in an ultrasonic probe 2 to emit pulsed ultrasonic signals into a subject to be examined (not shown). The pulsed ultrasonic signals are reflected in the subject to generate echoes that return to the vibrating elements 2a. The echoes are converted into electrical signals by the vibrating elements 2a, and the electrical signals are received by a receiver 5. The electrical signals representing the received echoes, i.e., echo signals, are input to a receive beamformer 6 where receive beamforming is performed. The receive beamformer 6 outputs receive-beamformed ultrasound data.

The receive beamformer 6 may be a hardware beamformer or a software beamformer. In the case that the receive beamformer 6 is a software beamformer, the receive beamformer 6 may comprise one or more processors including any one or more of a graphics processing unit (GPU), a microprocessor, a central processing unit (CPU), a digital signal processor (DSP), or other kinds of processors capable of executing logical operations. The processor(s) constituting the receive beamformer 6 may be constructed from a processor separate from a processor 7, which will be discussed later, or from the processor 7.

The ultrasonic probe 2 may comprise electrical circuitry to do all or part of the transmit and/or receive beamforming. For example, all or part of the transmit beamformer 3, transmitter 4, receiver 5, and receive beamformer 6 may be situated within the ultrasonic probe 2.

The ultrasonic diagnostic apparatus 1 also includes a processor 7 to control the transmit beamformer 3, transmitter 4, receiver 5, and receive beamformer 6. The processor 7 is in electronic communication with the ultrasonic probe 2. The processor 7 may control the ultrasonic probe 2 to acquire ultrasound data. The processor 7 controls which of the vibrating elements 2a are active, and the shape of an ultrasonic beam transmitted from the ultrasonic probe 2. The processor 7 is also in electronic communication with a display 8, and the processor 7 may process the ultrasound data into ultrasonic images for display on the display 8. The phrase “in electronic communication” may be defined to include both wired and wireless connections. The processor 7 may include a central processing unit (CPU) according to one embodiment. According to other embodiments, the processor 7 may include other electronic components capable of carrying out processing functions, such as a digital signal processor, a field-programmable gate array (FPGA), a graphics processing unit (GPU), or any other type of processor. According to other embodiments, the processor 7 may include a plurality of electronic components carrying out processing functions. For example, the processor 7 may include two or more electronic components selected from a list of electronic components including: a central processing unit, a digital signal processor, a field-programmable gate array, and a graphics processing unit.

The processor 7 may also include a complex demodulator (not shown) that demodulates RF data. In another embodiment, the demodulation can be carried out earlier in the processing chain.

The processor 7 is adapted to perform one or more processing operations according to a plurality of selectable ultrasonic modalities on the data. The data may be processed in real-time during a scanning session as the echo signals are received. For the purpose of this disclosure, the term “real-time” is defined to include a procedure that is performed without any intentional delay.

The data may be temporarily stored in a buffer (not shown) during ultrasonic scanning, so that they can be processed in a live operation or in an off-line operation not in real-time. In this disclosure, the term “data” may be used in the present disclosure to refer to one or more datasets acquired with an ultrasonic apparatus.

The ultrasound data (raw data) may be processed by other or different mode-related modules by the processor 7 (e.g., B-mode, color Doppler, M-mode, color M-mode, spectral Doppler, elastography, TVI, strain, strain rate, and the like) to form data for ultrasonic images. For example, one or more modules may produce ultrasonic images in B-mode, color Doppler, M-mode, color M-mode, spectral Doppler, elastography, TVI, strain, strain rate, and combinations thereof, and the like. The image beams and/or image frames are stored and timing information indicating a time at which the data was acquired in memory may be recorded. The modules may include, for example, a scan conversion module to perform scan conversion operations to convert the image frames from coordinate beam space to display space coordinates. A video processor module may be provided that reads the image frames from memory and displays the image frames in real-time while a procedure is being carried out on the subject. The video processor module may store the image frames in image memory, from which the ultrasonic images are read and displayed on the display 8.

In the case that the processor 7 includes a plurality of processors, the processing tasks to be handled by the processor 7 may be handled by the plurality of processors. For example, a first processor may be utilized to demodulate and decimate the RF signal while a second processor may be used to further process the data prior to displaying an image.

In the case that the receive beamformer 6 is a software beamformer, for example, its processing functions may be carried out by a single processor or by the plurality of processors.

The display 8 is an LED (Light Emitting Diode) display, an LCD (Liquid Crystal Display), an organic EL (Electro-Luminescence) display, or the like.

The memory 9 is any known data storage medium, and comprises non-transitory storage media and transitory storage media. The non-transitory storage media include, for example, a non-volatile storage medium such as an HDD (Hard Disk Drive), ROM (Read Only Memory), and the like. The non-transitory storage media may include a portable storage medium such as a CD (Compact Disk), a DVD (Digital Versatile Disk), and the like. Programs executed by the processor 7 are stored in a non-transitory storage medium.

Furthermore, the non-transitory storage medium constituting the memory 9 stores therein a machine learning algorithm.

The transitory storage medium is a volatile storage medium such as RAM (Random Access Memory), and the like.

The user interface 10 can accept an operator's input. For example, the user interface 10 accepts an input of a command and information from a user. The user interface 10 is adapted to include a keyboard, hard keys, a trackball, a rotary control, soft keys, and the like. The user interface 10 may include a touch screen that displays soft keys and the like.

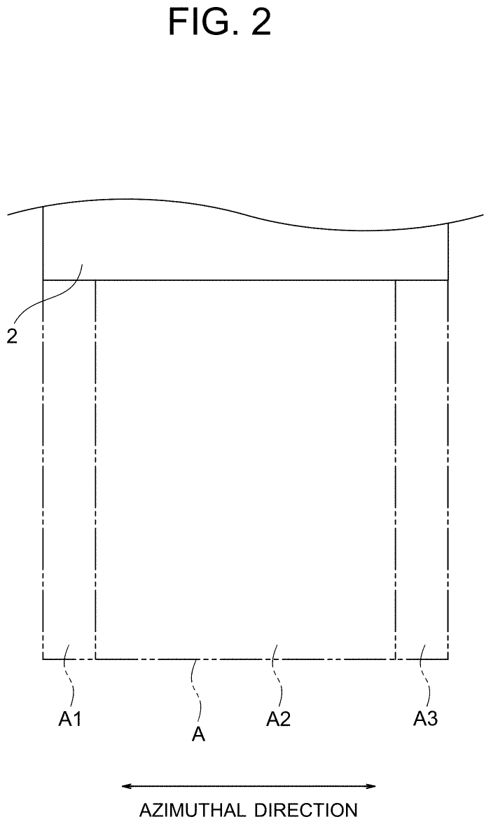

Next, an operation of the ultrasonic diagnostic apparatus in the present embodiment will be described. The ultrasonic probe 2 transmits ultrasonic beams to an image production region A in the subject shown in FIG. 2, and acquires echo signals. The image production region A is comprised of a first region A1, a second region A2, and a third region A3. The first region A1 and third region A3 lie on both end sides in an azimuthal direction, which is a direction of the width of the ultrasonic beam, in the image production region A. The second region A2 lies between the first region A1 and the third region A3.

The processor 7 controls transmission of ultrasonic beams BM to the image production region A by the ultrasonic probe 2, and production of an ultrasonic image of the image production region A based on echo signals. The processor 7 controls the ultrasonic probe 2 to transmit first and third focused ultrasonic beams BM1, BM3 having focus points F to the first region A1 and third region A3. The processor 7 also controls the ultrasonic probe 2 to transmit second ultrasonic beams BM2 that are plane waves to the second region A2.

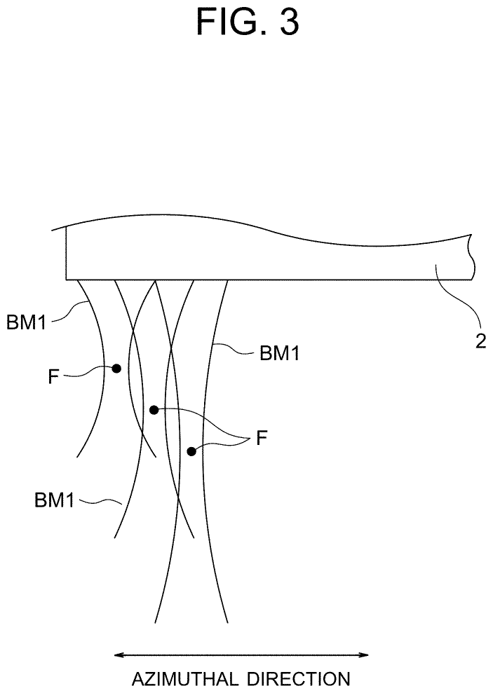

The processor 7 drives the plurality of elements 2a in the ultrasonic probe 2 via the transmit beamformer 3 and transmitter 4 to thereby transmit first and third ultrasonic beams BM1, BM3 having focus points. Specifically, the first and third ultrasonic beams BM1, BM3 are focused ultrasonic beams. FIG. 3 shows an example of the first ultrasonic beam BM1. As shown in FIG. 3, a plurality of the first ultrasonic beams BM1 are transmitted to different positions in the azimuthal direction. The plurality of first ultrasonic beams BM1 have respective focus points F at different positions in an acoustic-line direction. Similarly to the first ultrasonic beams BM1, the third ultrasonic beams BM3 are also transmitted to a plurality of different positions in the azimuthal direction, and have respective focus points F at different positions in the acoustic-line direction, although not particularly shown.

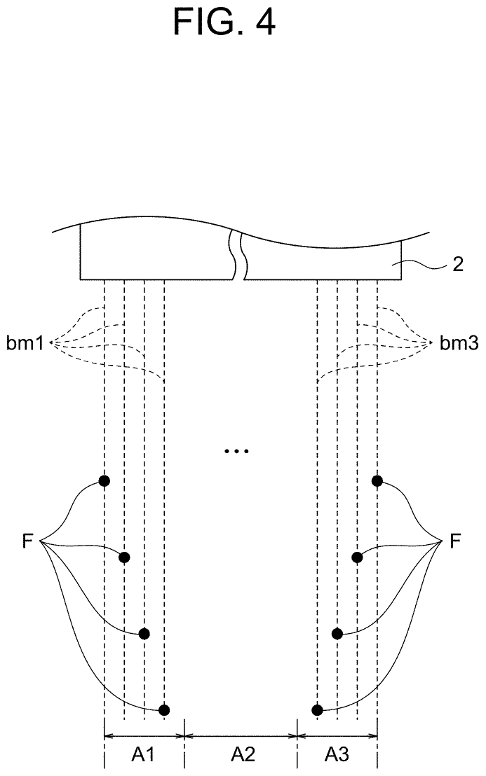

The positional relationship among the plurality of focus points F in the acoustic-line direction will now be described with reference to FIG. 4. In FIG. 4, dashed lines bm1, bm3 represent centers of the first and third ultrasonic beams BM1, BM3 in the direction of their widths (transmit acoustic lines). Among the plurality of focus points F shown on the dashed lines bm1, bm3, focus points F of the first and third ultrasonic beams BM1, BM3 that are closest to the second region are set at positions farthest away from the ultrasonic probe 2 (deepest positions). The first and third ultrasonic beams BM1, BM3 farther away from the second region have focus points F at positions closer to the ultrasonic probe 2 (shallower positions).

In the second region A2, a second ultrasonic beam having a focus point at infinity is transmitted, as will be discussed below. Therefore, by setting the focus points in the first and third regions A1, A3 to have the aforementioned positional relationship, a distance in the acoustic-line direction can be reduced between the position of the focus point of the second region A2 at infinity and the positions of the focus points of the first and third regions A1, A3. Thus, a border of image quality between a second ultrasonic image in the second region A2 and first and third ultrasonic images in the first and third regions A1, A3 becomes invisible, and an ultrasonic image having good image quality can be obtained.

Next, the second ultrasonic beam BM2 will be described. The processor 7 drives the plurality of elements 2a in the ultrasonic probe 2 via the transmit beamformer 3 and transmitter 4 to thereby transmit the second ultrasonic beam BM2. The processor 7 drives the plurality of elements 2a to form a plane wave and to transmit the second ultrasonic beam BM2 having the focus point at infinity.

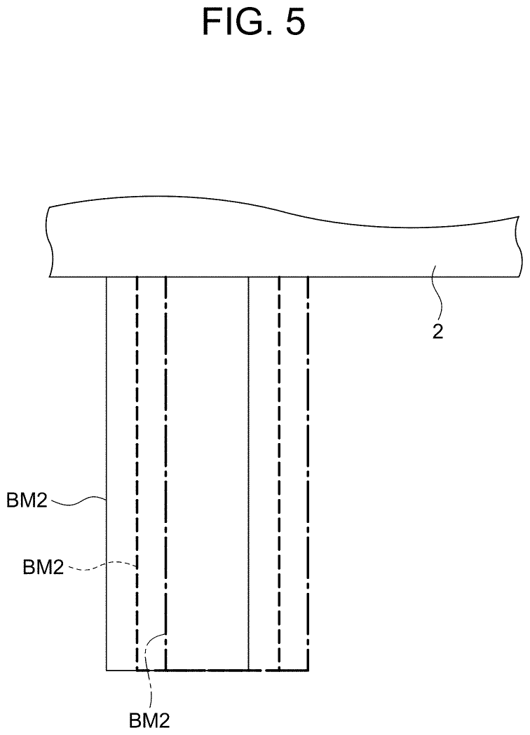

As shown in FIG. 5, a plurality of partially overlapping second ultrasonic beams BM2 are transmitted as the second ultrasonic beam BM2. In other words, the second ultrasonic beams BM2 are transmitted so that their respective transmit sound fields partially overlap one another.

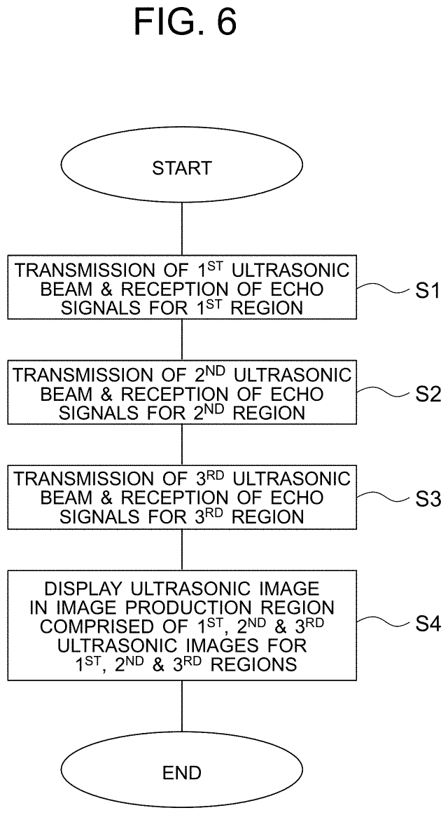

The flow of transmission of the ultrasonic beams BM and production of an ultrasonic image will now be described with reference to the flow chart in FIG. 6. First, at Step S1, the processor 7 controls the ultrasonic probe 2 to transmit the first ultrasonic beam BM1 to the first region A1. The ultrasonic probe 2 also receives echo signals from the first ultrasonic beam BM1.

The processor 7 creates raw data based on the echo signals from the first ultrasonic beam BM1. The processor 7 creates raw data in one or more receive acoustic lines falling within a transmit sound field of one first ultrasonic beam BM1 for storage in the memory 9. The number of receive acoustic lines for which raw data is created by transmission of one first ultrasonic beam BM1 is smaller than that for which raw data is created by transmission of one second ultrasonic beam BM2.

Next, at Step S2, the processor 7 controls the ultrasonic probe 2 to transmit the second ultrasonic beam BM2 to the second region A2. The ultrasonic probe 2 also receives echo signals from the second ultrasonic beam BM2. The processor 7 creates raw data in a receive acoustic line in the second region A2 based on the echo signals from the second ultrasonic beam BM2 for storage in the memory 9. Details thereof will be discussed later.

Next, at Step S3, the processor 7 controls the ultrasonic probe 2 to transmit the third ultrasonic beam BM3 to the third region A3. The ultrasonic probe 2 also receives echo signals from the third ultrasonic beam BM3.

The processor 7 creates raw data based on the echo signals from the third ultrasonic beam BM3. The processor 7 creates raw data in one or more receive acoustic lines falling within a transmit sound field of one third ultrasonic beam BM3 for storage in the memory 9. The number of receive acoustic lines for which raw data is created by transmission of one third ultrasonic beam BM3 is smaller than that for which raw data is created by transmission of one second ultrasonic beam BM2.

Next, at Step S4, the processor 7 produces a first ultrasonic image for the first region A1, a second ultrasonic image for the second region A2, and a third ultrasonic image for the third region A3 based on the raw data for the first, second, and third regions, respectively. The processor 7 then displays an ultrasonic image comprised of the first, second, and third ultrasonic images on the display 8.

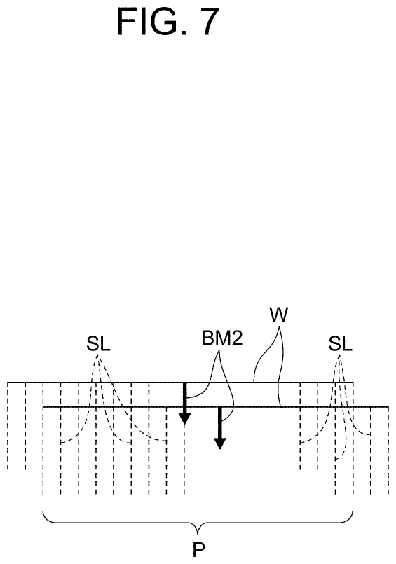

Creation of the raw data in receive acoustic lines in the second region A2 will now be described referring to FIG. 7. In FIG. 7, the second ultrasonic beams BM2 are indicated by arrows at respective center positions of the beam widths. Here are shown two second ultrasonic beams BM2. The length of each straight line indicated by symbol W represents the width of the transmit sound field for the second ultrasonic beam BM2.

The processor 7 creates raw data in receive acoustic lines SL falling within the width W of the transmit sound field. The width W of the transmit sound field for one second ultrasonic beam BM2 contains a plurality of receive acoustic lines SL.

As described with reference to FIG. 5, the plurality of second ultrasonic beams BM2 have respective transmit sound fields partially overlapping one another in the second region A2. Therefore, for one receive acoustic line SL, raw data corresponding respectively to the plurality of second ultrasonic beams BM2 can be obtained. For example, in FIG. 7, two second ultrasonic beams BM2 have respective transmit sound fields overlapping in a portion P, so that raw data corresponding respectively to the two second ultrasonic beams BM2 are obtained in a receive acoustic line in the portion P.

The processor 7 adds together raw data corresponding respectively to the plurality of second ultrasonic beams BM2 having overlapping transmit sound fields to obtain raw data in one receive acoustic line used in production of an ultrasonic image. The creation of raw data in the second region is similar to the publicly known RTF method.

In acquiring raw data by transmitting ultrasonic beams that are plane waves for the entire image production region in a similar manner to the publicly known RTF method, the amount of raw data to be added together, and the number of the elements 2a used in the transmission and reception are different depending upon the position of a receive acoustic line: This will now be explained hereinbelow.

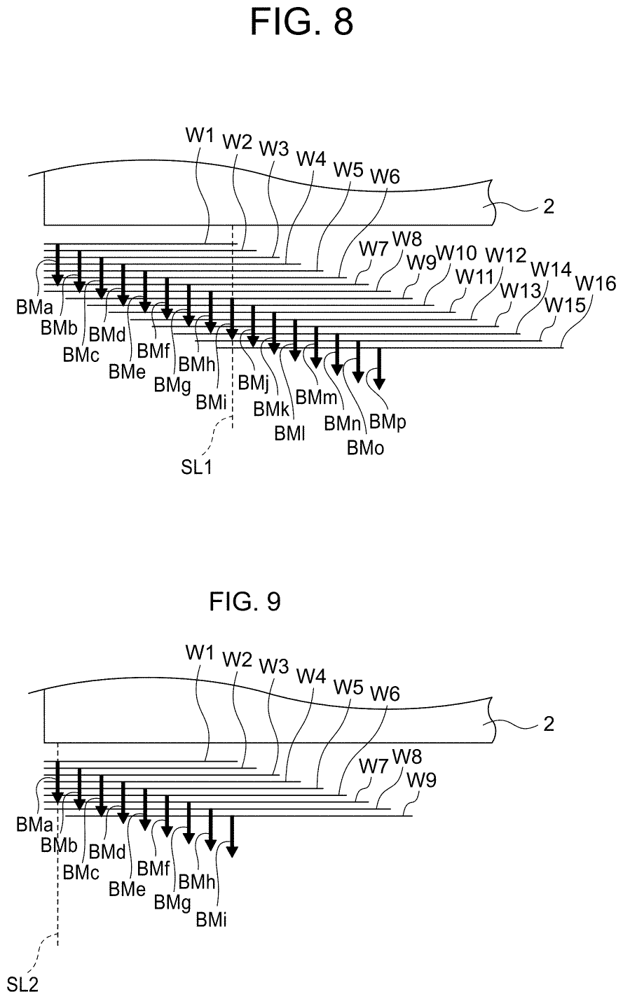

First, creation of raw data in a receive acoustic line SL1 shown in FIG. 8 will be described. In FIG. 8, ultrasonic beams BMa to BMp having respective widths W1 to W16 of the transmit sound fields are shown. The receive acoustic line SL1 falls within the widths W1 to W16 of the transmit sound fields. Therefore, raw data for image production in the receive acoustic line SL1 is obtained by adding together raw data corresponding respectively to the plurality of ultrasonic beams BMa to BMp.

Next, creation of raw data in a receive acoustic line SL2 shown in FIG. 9 will be described. The receive acoustic line SL2 lies closer to an end portion of the image production region than the receive acoustic line SL1 shown in FIG. 8. Although the receive acoustic line SL2 falls within the widths W1 to W8 of the transmit sound fields, it falls outside of the width W9 of the transmit sound field of an ultrasonic beam BMi transmitted next to an ultrasonic beam BMh. Therefore, raw data for image production in the receive acoustic line SL2 is obtained by adding together raw data corresponding respectively to the plurality of ultrasonic beams BMa to BMh.

Hence, the amount of raw data to be added together in creation of raw data for image production in the receive acoustic line SL2 is smaller than that in the receive acoustic line SL1. Moreover, since the ultrasonic beams BMa to BMh are those lying near the end portion of the ultrasonic probe 2, the widths W1 to W8 of the transmit sound fields are smaller than the widths W9 to W16 of the transmit sound fields. Accordingly, the number of the elements 2a used in the transmission/reception for obtaining raw data in the receive acoustic line SL2 is smaller than that in the receive acoustic line SL1 as well. From the facts above, in transmitting ultrasonic beams that are plane waves in the entire image production region, the receive acoustic line SL2 has a lower S/N ratio than the receive acoustic line SL1.

From the reason above, assuming that plane waves are transmitted also in the first and third regions A1, A3 as in the publicly known RTF method, first and third ultrasonic images obtained in the first and third regions A1, A3 have a lower S/N ratio than a second ultrasonic image obtained in the second region A2. In the present embodiment, in the first and third regions A1, A3, first and third focused ultrasonic beams BM1, BM3 are transmitted, whereby the S/N ratio for the first and third ultrasonic images can be improved as compared with the case in which plane waves are transmitted.

Moreover, since the second ultrasonic beams BM2 that are plane waves are transmitted in the second region A2, the frame rate can be improved as compared with the case in which focused ultrasonic beams are transmitted in the entire image production region.

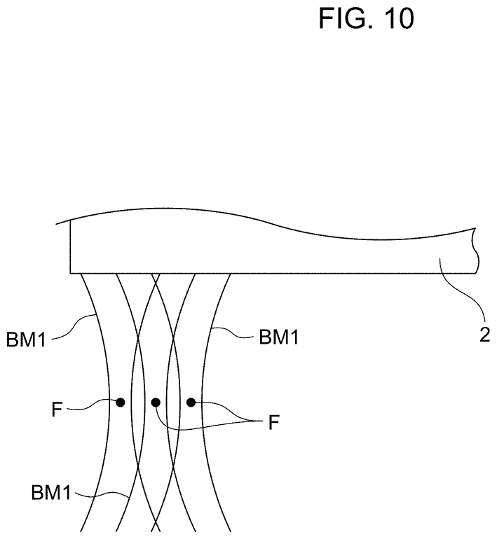

Next, a variation of the embodiment will be described. As shown in FIG. 10, the processor 7 may control the ultrasonic probe 2 via the transmit beamformer 3 and transmitter 4 so that the positions of the respective focus points F of the plurality of first ultrasonic beams BM1 in the acoustic-line direction are identical. Moreover, the processor 7 may control the ultrasonic probe 2 via the transmit beamformer 3 and transmitter 4 so that the positions of the respective focus points F of the plurality of first ultrasonic beams BM3 in the acoustic-line direction are identical, similarly to the first ultrasonic beam BM1, although not particularly shown.

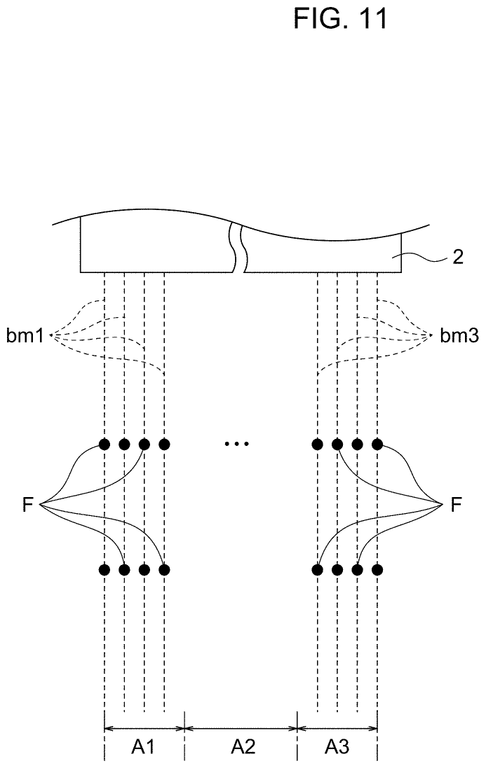

Furthermore, as shown in FIG. 11, the processor 7 may control the ultrasonic probe 2 via the transmit beamformer 3 and transmitter 4 so that a plurality of focus points F are formed for one transmit acoustic line, as indicated by dashed lines bm1, bm3, in the first and third regions A1, A3. Specifically, the processor 7 may control the ultrasonic probe 2 to transmit a plurality of first ultrasonic beams BM1 having different positions of focus points F for one transmit acoustic line, and transmit a plurality of third ultrasonic beams BM3 having different positions of focus points F for one transmit acoustic line. In FIG. 11, two focus points F are set for one transmit acoustic line, and two first and third ultrasonic beams BM1, BM3 are transmitted for one transmit acoustic line. By thus setting a plurality of focus points F for one transmit acoustic line, lowering of penetration can be prevented as compared with the case in which a single focus point is set in a relatively shallow portion.

Additionally, the processor 7 may perform different processing between signal processing on first and third echo signals and that on second echo signals. For example, the processor 7 may employ a different receive filter in signal processing on first and third echo signals from that employed in signal processing on second echo signals so that lowering of penetration due to the focus points in the first and third regions A1, A3 set at relatively shallow positions than in the second region A2 is compensated. Moreover, the processor 7 may employ a different gain in signal processing on first and third echo signals from that employed in signal processing on second echo signals so that lowering of brightness accompanying the aforementioned lowering of penetration is compensated.

While the present invention has been described with reference to the embodiment above, it will be easily appreciated that the present invention may be practiced with several modifications without departing from the spirit and scope thereof. For example, while the order of transmissions of the first, second, and third ultrasonic beams BM1, BM2, BM3 in the embodiment above is not limited to that shown in the flow chart in FIG. 6. Moreover, transmission of the first ultrasonic beam BM1 in one or some transmit acoustic lines in the first region A1, transmission of the second ultrasonic beam BM2 in one or some transmit acoustic lines in the second region A2, and transmission of the third ultrasonic beam BM3 in the third region A3 may be repeated in this order.

Furthermore, the embodiment described above may be a method of controlling an ultrasonic apparatus comprising:

an ultrasonic probe for transmitting ultrasonic beams to an image production region in a subject to be examined and acquiring echo signals, wherein said image production region is comprised of a first region, a second region, and a third region, said first region and said third region lying on both end sides in an azimuthal direction orthogonal to a direction of transmission of said ultrasonic beams, and said second region lying between said first region and said third region; and

a processor for controlling transmission of said ultrasonic beams by said ultrasonic probe, and production of an ultrasonic image for said image production region based on said echo signals, wherein said processor

controls said ultrasonic probe to transmit a first ultrasonic beam to said first region, transmit a second ultrasonic beam to said second region, and transmit a third ultrasonic beam to said third region, said first and third ultrasonic beams being focused ultrasonic beams, and said second ultrasonic beam being an ultrasonic beam formed by a plane wave, and

produces said ultrasonic image comprised of a first ultrasonic image for said first region, a second ultrasonic image for said second region, and a third ultrasonic image for said third region based on first, second, and third echo signals obtained from said first, second, and third regions by transmissions of said first, second, and third ultrasonic beams.

This written description uses examples to disclose the present invention, including the best mode, and also to enable any person skilled in the art to practice the present invention, including making and using any computing system or systems and performing any incorporated methods. The patentable scope of the present invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An ultrasonic apparatus comprising:
an ultrasonic probe for transmitting ultrasonic beams to an image production region in a subject to be examined and acquiring echo signals, wherein said image production region is comprised of a first region, a second region, and a third region, said second region lying between said first region and said third region; and
a processor for controlling transmission of said ultrasonic beams by said ultrasonic probe, and production of an ultrasonic image for said image production region based on said echo signals, wherein said processor
controls said ultrasonic probe to transmit a first ultrasonic beam to said first region, transmit a second ultrasonic beam to said second region, and transmit a third ultrasonic beam to said third region, said first and third ultrasonic beams being focused ultrasonic beams, and said second ultrasonic beam being a plane wave, and
produces said ultrasonic image comprised of a first ultrasonic image for said first region, a second ultrasonic image for said second region, and a third ultrasonic image for said third region based on first, second, and third echo signals obtained from said first, second, and third regions by transmissions of said first, second, and third ultrasonic beams.

2. The ultrasonic apparatus as recited in claim 1, wherein: said processor controls said ultrasonic probe to transmit a plurality of said first ultrasonic beams along different acoustic-lines in said first region and a plurality of said third ultrasonic beams along different acoustic-lines in said third region, the plurality of said first ultrasonic beams having respective focus points at different positions in an acoustic-line direction, and the plurality of said third ultrasonic beams having focus points at different positions in the acoustic-line direction.

3. The ultrasonic apparatus as recited in claim 2, wherein: the position of said focus point in the acoustic-line direction is deeper for first and third ultrasonic beams closer to said second region.

4. The ultrasonic apparatus as recited in claim 1, wherein: said processor controls said ultrasonic probe to transmit a plurality of said first ultrasonic beams along different acoustic-lines in said first region and a plurality of said third ultrasonic beams along different acoustic-lines in said third region, the plurality of said first ultrasonic beams having respective focus points at an identical position in the acoustic-line direction, and the plurality of said third ultrasonic beams each having a focus point at an identical position in the acoustic-line direction.

5. The ultrasonic apparatus as recited in claim 1, wherein: said processor controls said ultrasonic probe to transmit a plurality of ultrasonic beams having partially overlapping transmit sound fields as said second ultrasonic beam, and for a receive acoustic line in a portion in which said transmit sound fields overlap, creates data used in production of said second ultrasonic image by adding together raw data obtained by transmission of the plurality of second ultrasonic beams having the overlapping transmit sound fields.

6. The ultrasonic apparatus as recited in claim 2, wherein: said processor controls said ultrasonic probe to transmit a plurality of ultrasonic beams having partially overlapping transmit sound fields as said second ultrasonic beam, and for a receive acoustic line in a portion in which said transmit sound fields overlap, creates data used in production of said second ultrasonic image by adding together raw data obtained by transmission of the plurality of second ultrasonic beams having the overlapping transmit sound fields.

7. The ultrasonic apparatus as recited in claim 3, wherein: said processor controls said ultrasonic probe to transmit a plurality of ultrasonic beams having partially overlapping transmit sound fields as said second ultrasonic beam, and for a receive acoustic line in a portion in which said transmit sound fields overlap, creates data used in production of said second ultrasonic image by adding together raw data obtained by transmission of the plurality of second ultrasonic beams having the overlapping transmit sound fields.

8. The ultrasonic apparatus as recited in claim 4, wherein: said processor controls said ultrasonic probe to transmit a plurality of ultrasonic beams having partially overlapping transmit sound fields as said second ultrasonic beam, and for a receive acoustic line in a portion in which said transmit sound fields overlap, creates data used in production of said second ultrasonic image by adding together raw data obtained by transmission of the plurality of second ultrasonic beams having the overlapping transmit sound fields.

9. The ultrasonic apparatus as recited in claim 1, wherein: said processor controls said ultrasonic probe to transmit in said first region a plurality of said first ultrasonic beams each having focus points at different positions in one transmit acoustic line, and transmit in said third region a plurality of said third ultrasonic beams each having focus points at different positions in one transmit acoustic line.

10. The ultrasonic apparatus as recited in claim 1, wherein: said processor performs different processing between signal processing on said first and third echo signals, and that on said second echo signals.

11. The ultrasonic apparatus as recited in claim 10, wherein: said processor uses receive filter and gain in the signal processing on said first and third echo signals different from those used in the signal processing on said second echo signals.

12. The ultrasonic apparatus as recited in claim 11, wherein: said processor uses receive filter and gain for compensating lowering of penetration as the receive filter and gain used in the signal processing on said first and third echo signals.

13. The ultrasonic apparatus as recited in claim 1, wherein: said processor creates raw data in one or more receive acoustic lines from echo signals acquired by transmission of said first and third ultrasonic beams to produce said first and third ultrasonic images.

14. A method of controlling an ultrasonic apparatus comprising:
transmitting a first ultrasonic beam, a second ultrasonic beam and a third ultrasonic beam to an image production region in a subject to be examined, wherein said first ultrasonic beam is a focused ultrasound beam and is transmitted to a first region of said image production region, wherein said second ultrasonic beam is a plane wave and is transmitted to a second region of said image production region, wherein said third ultrasonic beam is a focused ultrasound beam and is transmitted to a third region of said image production region, said second region lying between said first region and said third region;
acquiring a first echo signal of said first ultrasonic beam, a second echo signal of said second ultrasonic beam and a third echo signal of said third ultrasonic beam; and
producing ultrasonic image comprised of a first ultrasonic image for said first region, a second ultrasonic image for said second region, and a third ultrasonic image for said third region based on said first, said second, and said third echo signals.

15. The method as recited in claim 14, wherein: a plurality of said first ultrasonic beams are transmitted along different acoustic-lines in said first region and a plurality of said third ultrasonic beams are transmitted along different acoustic-lines in said third region, the plurality of said first ultrasonic beams having respective focus points at different positions in an acoustic-line direction, and the plurality of said third ultrasonic beams having focus points at different positions in the acoustic-line direction.

16. The method as recited in claim 15, wherein: the position of said focus point in the acoustic-line direction is deeper for first and third ultrasonic beams closer to said second region.

17. The method as recited in claim 14, wherein: a plurality of said first ultrasonic beams are transmitted along different acoustic-lines in said first region and a plurality of said third ultrasonic beams are transmitted along different acoustic-lines in said third region, the plurality of said first ultrasonic beams having respective focus points at an identical position in the acoustic-line direction, and the plurality of said third ultrasonic beams each having a focus point at an identical position in the acoustic-line direction.

18. The method as recited in claim 14, wherein: a plurality of said second ultrasonic beams having partially overlapping transmit sound fields are transmitted, and further comprising:
acquiring echo signals of said second ultrasonic beams from a receive acoustic line in a portion in which said transmit sound fields overlap;
wherein said second ultrasonic image is created by adding together echo signals in said receive acoustic line.

19. The method as recited in claim 14, wherein: a plurality of said first ultrasonic beams each having focus points at different positions are transmitted in one transmit acoustic line, and a plurality of said third ultrasonic beams each having focus points at different positions are transmitted in one transmit acoustic line.

20. The method as recited in claim 14, wherein: said processor uses receive filter and gain in the signal processing on said first and third echo signals different from those used in the signal processing on said second echo signals.
